(19) United States
(12) Patent Application Publication
(10) Pub. No.: US 2010/0242961 A1
(43) Pub. Date: Sep. 30, 2010

(54) SYSTEMS AND METHODS FOR PREVENTING WATER DAMAGE IN A BREATHING ASSISTANCE SYSTEM

(75) Inventors: Laurent Mougel, Sainte Marguerite (FR); Arnaud Mocellin, Toul (FR); Philippe Perine, Eulont (FR); Hossein Nadjafizadeh, Villers-Les-Nancy (FR)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/415,162

(22) Filed: Mar. 31, 2009

(51) Int. Cl. A61M 16/16

(52) U.S. Cl. 128/203.16

(57) ABSTRACT

A breathing assistance system includes a gas delivery system including a gas flow generation device configured to generate a gas flow; a humidifier system including a liquid water chamber and an outlet, and a microporous filter. The gas delivery system and the humidifier system at least partially define a gas flow path from the gas flow generation device to the outlet of the humidifier system. The microporous filter is located along the gas flow path such that the gas flow generated by the gas flow generation device flows through the microporous filter, subsequently becomes humidified in the liquid water chamber, and subsequently flows through the outlet of the humidifier system. The microporous filter is gas-permeable and liquid water-impermeable, such that the microporous filter prevents liquid water in the liquid water chamber from flowing into the gas flow generation device in any orientation of the gas delivery system and humidifier system.

### TECHNICAL FIELD

The invention relates to humidifiers, e.g., systems and methods for preventing water damage in a breathing assistance system (e.g., ventilator or CPAP device).

### BACKGROUND

Many breathing assistance systems (e.g., CPAP devices, mechanical ventilators, etc.) use humidifiers in order to provide humidified air to a patient. Humidification may prevent various conditions, e.g., hypothermia, inspissation of airway secretions, destruction of airway epithelium, and atelectasis.

Humidifiers can be passive or active. Passive humidifiers (e.g., a heat-and-moisture exchanger (HME), which may be referred to as an “artificial nose”) may trap heat and humidity from the patient's exhaled gas and return some of the trapped heat and humidity to the patient during the subsequent inhalation. Active, or heated, humidifiers typically pass the inspired gas through or over a heated water bath to increase the heat and water vapor content of the inspired gas.

In certain breathing assistance systems, a humidifier shares a housing with, or is otherwise physically integrated with, a gas delivery system (e.g., a motorized blower, piston-based device, flow-control valves, a compressor, etc.). In such systems, liquid water from the humidifier may flow into the gas flow generation system when the system is tilted, turned on its side, or turned over, which may damage components of the gas delivery system (e.g., a blower, motor, electronics, etc.) and/or other components of the breathing assistance system.

### SUMMARY

In accordance with one embodiment of the present disclosure, a breathing assistance system includes a gas delivery system including a gas flow generation device configured to generate a gas flow; a humidifier system including a liquid water chamber and an outlet, and a microporous filter. The gas delivery system and the humidifier system at least partially define a gas flow path from the gas flow generation device to the outlet of the humidifier system. The microporous filter is located along the gas flow path such that the gas flow generated by the gas flow generation device flows through the microporous filter, subsequently becomes humidified in the liquid water chamber, and subsequently flows through the outlet of the humidifier system. The microporous filter is gas-permeable and liquid water-impermeable, such that the microporous filter prevents liquid water in the liquid water chamber from flowing into the gas flow generation device in any orientation of the gas delivery system and humidifier system.

In accordance with another embodiment of the present disclosure, a method is provided for providing humidification for a breathing assistance system including a gas delivery system including a gas flow generation device, a humidifier system including a liquid water chamber and an outlet, and a gas-permeable, liquid water-impermeable microporous filter located along a gas flow path from the gas flow generation device to the humidifier system outlet. The method includes generating a gas flow using the gas flow generation device; communicating the gas flow from the gas flow generation device, through the gas-permeable/liquid water-impermeable microporous filter, and into the liquid water chamber; humidifying the gas flow in the liquid water chamber; and communicating the humidified gas flow through the humidifier system outlet.

In accordance with another embodiment of the present disclosure, a breathing assistance system includes a gas flow generation device configured to generate a gas flow, a humidifier, and a microporous filter located between the gas flow generation device and the humidifier such that the gas flow generated by the gas flow generation device flows through the microporous filter, subsequently becomes humidified in a chamber of the humidifier, and subsequently flows through an outlet of the humidifier. The microporous filter is gas-permeable and liquid water-impermeable, such that the microporous filter prevents liquid in the humidifier from flowing to the gas flow generation device in any orientation of the breathing assistance system.

### BRIEF DESCRIPTION OF THE DRAWINGS

For a more complete understanding of the present invention, and the advantages thereof, reference may be made to the following description of exemplary embodiments, taken in conjunction with the accompanying drawings, in which:

FIG. 1 illustrates an example breathing assistance system for delivering humidified gas to a patient, including a microporous filter for preventing water damage in a gas delivery system, according to certain embodiments of the disclosure;

FIG. 2A illustrates an example breathing assistance system including a microporous filter located between a gas delivery system and a humidifier system, in an upright orientation, according to certain embodiments of the disclosure;

FIG. 2B illustrates the example breathing assistance system of FIG. 2A in an upside-down orientation, illustrating the function of the microporous filter, according to certain embodiments of the disclosure;

FIG. 3A illustrates an example breathing assistance system including a microporous filter located at least partially within a gas delivery system, in an upright orientation, according to certain embodiments of the disclosure;

FIG. 3B illustrates the example breathing assistance system of FIG. 3A in an upside-down orientation, illustrating the function of the microporous filter, according to certain embodiments of the disclosure;

FIG. 4A illustrates an example breathing assistance system including a microporous filter located at least partially within a humidifier system, in an upright orientation, according to certain embodiments of the disclosure;

FIG. 4B illustrates the example breathing assistance system of FIG. 4A in an upside-down orientation, illustrating the function of the microporous filter, according to certain embodiments of the disclosure;

FIG. 5 illustrates an example breathing assistance system including an integrated gas delivery system and a humidifier system, according to certain embodiments of the disclosure; and

FIG. 6 illustrates a method for protecting a gas delivery system from water damage using a microporous filter, according to certain embodiments of the disclosure.

### DETAILED DESCRIPTION OF THE DRAWINGS

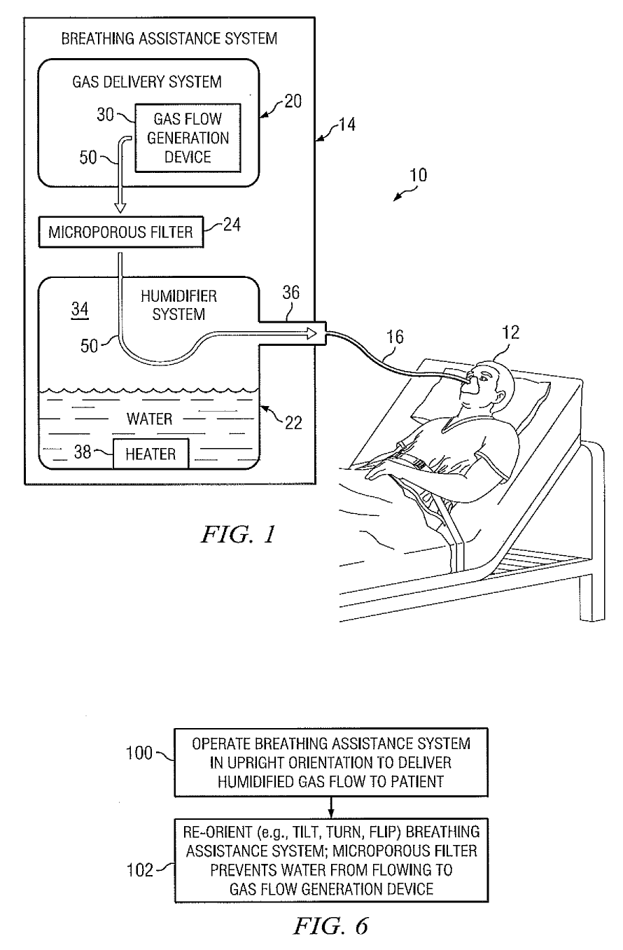

Selected embodiments of the disclosure may be understood by reference, in part, to FIGS. 1-6, wherein like numbers refer to same and like parts. The present disclosure relates generally to breathing assistance systems including integrated or attachable humidifiers, e.g., for providing CPAP therapy, ventilation, or other breathing assistance to patients. A breathing assistance system (e.g., a CPAP device or ventilator) may include a gas delivery system for generating a gas flow (e.g., a pressurized air flow) and a humidifier system for humidifying the gas flow before the gas is delivered to a patient, e.g., via a patient circuit. A microporous filter is located such that the gas flow generated by the gas delivery system through the microporous filter and into a liquid water chamber of the humidifier system, becomes humidified by water in the liquid water chamber, flows out through a humidifier system outlet, and is delivered to the patient via a patient circuit or other connection.

The microporous filter is gas-permeable and liquid water-impermeable, such that the microporous filter prevents liquid water in the humidifier system from flowing into the gas delivery system, regardless of the physical orientation of the gas delivery system and humidifier system (e.g., upright, tilted, on its side, upside-down, etc.). Thus, the microporous filter may protect components of the gas delivery system (e.g., a blower, motor, electronics, etc.) from being damaged by water. In some embodiments, the microporous filter is located between the gas delivery system and the humidifier system. In other embodiments, the microporous filter is located at least partially within either the gas delivery system or the humidifier system. In other embodiments, the microporous filter extends into both the gas delivery system and the humidifier system.

FIG. 1 illustrates an example system 10 for delivering humidified gas to a patient 12, according to one embodiment of the disclosure. System 10 may include a breathing assistance system 14 for generating a humidified gas flow, and a connection system 16 for delivering the humidified gas flow to patient 12.

Breathing assistance system 14 may comprise any device, apparatus, or system for generating a humidified gas flow to be delivered to a patient, e.g., a ventilator, a respirator, a CPAP/Auto CPAP device, or a BiPAP/Auto BiPAP device. Breathing assistance system 14 may include a gas delivery system 20, a humidifier system 22, and a microporous filter 24. Gas delivery system 20 may include a gas flow generation device 30 configured to generate, supply, and/or deliver gas (e.g., pressurized air) toward patient 12. For example, gas flow generation device 30 may comprise a device capable of generating pressurized air (e.g., a motorized blower or piston-based device), a wall outlet through which pressurized air may be supplied (e,g., in a hospital or clinic) and/or conduits for communicating air from a wall outlet, valves configured to control the supply of gas to the patient (e,g., a PSOL or other solenoid valve), one or more tanks of compressed gas, a compressor, and/or any other suitable source of pressurized or non-pressurized gas. In certain embodiments, gas flow generation device 30 includes a blower including an electric motor and other suitable electronics.

As used herein, the term “gas” may refer to any one or more gases and/or vaporized substances suitable to be delivered to and/or from a patient via one or more breathing orifices (e.g., the nose and/or mouth), such as air, nitrogen, oxygen, any other component of air, CO₂, vaporized water, vaporized medicines, and/or any combination of two or more of the above, for example.

As used herein, the term “patient” may refer to any person or animal that may receive breathing assistance from system 10, regardless of the medical status, official patient status, physical location, or any other characteristic of the person. Thus, for example, patients may include persons under official medical care (e.g., hospital patients), persons not under official medical care, persons receiving care at a medical care facility, persons receiving home care, etc.

Humidifier system 22 may be generally operable to humidify (e.g., to increase the heat and/or water vapor content) a gas flow from gas delivery system 20 to then be delivered to patient 12 via connection system 16. Humidifier system 22 may or may not be a heated humidifier. Humidifier system 22 may be permanently or removably attached to gas delivery system 20. In some embodiments, humidifier system 22 is physically integrated with gas delivery system 20. In some embodiments, humidifier system 22 may be directly physically connected to gas delivery system 20. In other embodiments, humidifier system 22 may be indirectly connected to gas delivery system 20 via one or more gas delivery conduits 22 and/or via microporous filter 24.

Humidifier system 22 may include a liquid water chamber 34 configured to hold liquid water, and an outlet 36 for communicating humidified gas to connection system 16. Liquid water chamber 34 may have any suitable shape and configuration and may be configured to hold any suitable volume of liquid water. In embodiments in which humidifier system 22 is a heated humidifier, humidifier system 22 may include a heater 38 and any suitable electronics (an electrical, gas, or battery-powered heating device). Humidifier system 22 may be directly or indirectly coupled to connection system 16 in any suitable manner. In some embodiments, outlet 36 defines an outlet from breathing assistance system 14, such that connection system 16 may be coupled directly to outlet 36. In other embodiments (e.g., as shown in FIG. 5), outlet 36 of humidifier system 22 may open to one or more other internal chambers or conduits of breathing assistance system 14, which may in turn lead to an outlet from breathing assistance system 14 (e.g., outlet 60 shown in FIG. 5) to which connection system 16 may be connected.

Microporous filter 24 is located along the gas flow path of breathing assistance system 14 such that the gas flow generated by gas flow generation device 30 flows through microporous filter 24, becomes humidified by humidifier system 22, and flows through outlet 36 and into connection system 16 toward patient 12. In certain embodiments, microporous filter 24 is gas-permeable and liquid water-impermeable, such that filter 24 prevents liquid water in liquid water chamber 34 from flowing into gas flow generation device 30 in any physical orientation of the gas delivery system and humidifier system, e.g., upright, tilted, on its side, upside-down, etc. In this manner, microporous filter 24 may protect components of gas delivery system 24 (e.g., a blower, motor, electronics, etc.) from being damaged by water.

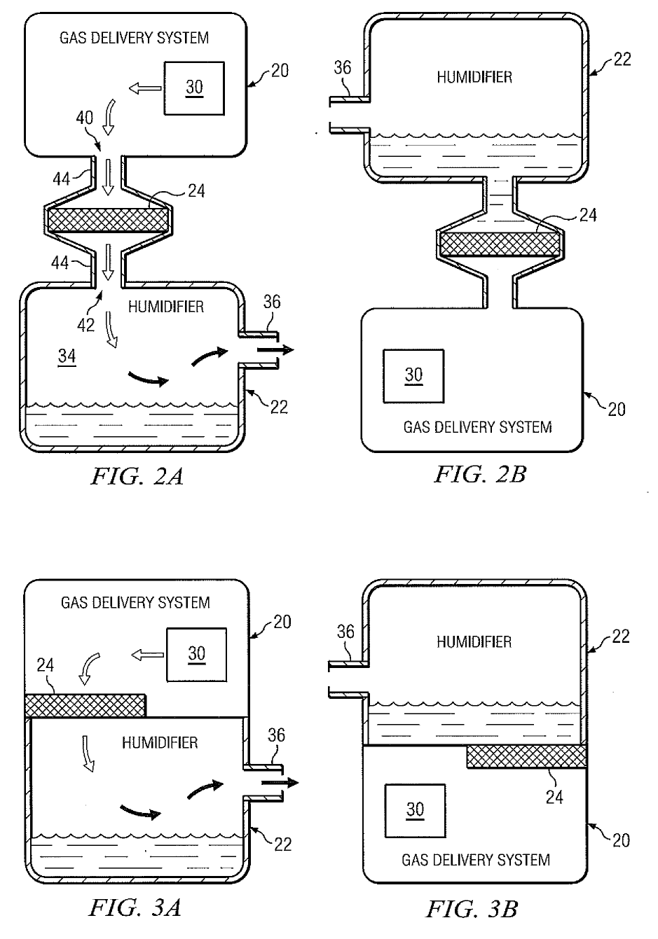

In some embodiments (e.g., FIGS. 1A-1B), microporous filter 24 is located between gas delivery system 30 and humidifier system 22. In other embodiments (e.g., FIGS. 2A-2B), microporous filter 24 is located at least partially within gas delivery system 30. In still other embodiments (e.g., FIGS. 3A-3B), microporous filter 24 is located at least partially within humidifier system 22. In still other embodiments, microporous filter 24 may extend into both gas delivery system 30 and humidifier system 22.

Microporous filter 24 may include any gas-permeable and liquid water-impermeable filter, membrane, or material. For example, microporous filter 24 may include a polypropylene filter or membrane. Microporous filter 24 may be supported or housed in any suitable housing or structure. In some embodiments, the filter housing may have a removable or accessible cover or other housing component that can be manipulated for accessing the filter material, e.g., to clean, replace, etc. the filter material.

In addition, microporous filter 24 may include an anti-bacterial membrane that filters the air flow coming from the gas delivery system 30 and/or from the patient 12. By using an anti-bacterial membrane, the same filter 24 may be used by several patients (e.g., in a sleep lab) by only changing the filter 24 when changing between patients.

Several examples of microporous filter 24 are provided below. It should be understood that these are examples only, and that microporous filter 24 may include any other gas-permeable, liquid water-impermeable structure.

Microporous Filter Example 1: Membrane GSB-70 by 3M Company™

3M™ Air Filter Media Type GSB are constructed from permanently charged polypropylene (PP) fibers able to capture airborne particles. The fibers used for GSB media are electrets split fibers. The fibers are bipolar charged to a typical level of about 50 nC/cm². In the cross-section the rectangular fibers have a thickness of about 10 μm and a width of about 40 μm. The fibers are carded and bonded by needling to yield an open and uniform non-woven web. This construction enables GSB media to capture particles throughout the entire media depth, rather than only on the surface. The construction includes 10 g/m² polypropylene spunbond scrim and 60 g/m² electret fiber, bonded by needling.

Various properties of the GSB-70 filter are provided in the following table.

| Property | Typical values |
|---|---|
| Total basis weight | 70 g/m² |
| Electret fiber weight | 60 g/m² |
| Penetration | 37.5% (0.1 μm NaCl @ 0.2 m/s measured on a |
| | TSI AFT 8130 sample area: 50 cm² |
| Pressure drop | 6.3 Pa (@ 0.2 m/s measured on a TSI AFT 8130 |
| | sample area: 50 cm² |
| Peel strength | >0.1 N/50 mm |
| RoHS compliance | All raw materials used in this media are RoHS |
| | compliant |

Microporous Filter Example 2: TECHNOSTAT XA Membrane by Hollingsworth & Vose Company (Used in DAR™ Filters)

TECHNOSTAT XA is available in grades from 23 g/m² to over 500 g/m². In some embodiments a grade of 250 g/m²±10% is used. The scrim weight may be 15 g/m²±10%. The composition of the product is blended synthetic fibre attached (laminated) to 15 g/m² polypropylene spunbonded scrim, for a thickness of 3.3 mm. The product has a BS4400 NcCl penetration of 1.1% at 9.5 m/min media velocity, and an air flow resistance of 31.4 Pa at 9.5 m/min media velocity. In some embodiments, additional material layers can be attached either during the needle felting operation or via a lamination process, which may enhance the physical properties or improve filtration performance of the filter.

Microporous Filter Example 3: Barrierbac Filter by NELLCOR™ (e.g., http://www.medcompare.com/details/41634/Barrierbac-Filter.html)

The Barrierbac Filter by NELLCOR™ is generally configured for use on anesthetized patients and respiratory care patients that use a breathing circuit. The filter includes electrostatic hydrophobic media that protects against cross-contamination. The filter may be placed at the patient side or the breathing assistance system side. The filter has relatively low resistance to air flow, which may reduce the patient's work of breathing in certain breathing assistance applications. The filter may include a standardized fitting (e.g., 15 mm or 22 mm) to connect to a standardized breathing circuit 16.

Various specifications of the Barrierbac Filter are provided in the following table.

| Property | Typical values |
|---|---|
| Resistance to flow (ISO 9360): | |
| 30 l/min | 0.65 cm H₂O |
| 60 l/min | 1.55 cm H₂O |
| 90 l/min | 2.80 cm H₂O |
| Bacterial filtration efficiency | 99.999% |
| Viral filtration efficiency | 99.999% |
| Dead space (connectors | 99 ml |
| included -- ISO 9360 | |
| Weight | 35 g |
| Type of filtration | Electrostatic |

Microporous Filter Example 4: Barrierbac S Filter by NELLCOR™ (e.g., http://www.bio-medicine.org/medicine-products/Barrierbac-S-Filter-16870-1/)

The Barrierbac S Filter by NELLCOR™ is generally configured for use on anesthetized patients and respiratory care patients that use a breathing circuit. The filter includes electrostatic hydrophobic media that protects against cross-contamination. The filter may be placed at the patient side or the breathing assistance system side. The filter includes an end tidal CO₂ sampling port for convenient access to monitor airway gases. The filter has relatively low resistance to air flow, which may reduce the patient's work of breathing in certain breathing assistance applications. The filter may include a standardized fitting (e.g., 15 mm or 22 mm) to connect to a standardized breathing circuit 16.

Various specifications of the Barrierbac Filter are provided in the following table.

| Property | Typical values |
|---|---|
| Resistance to flow (ISO 9360): | |
| 30 l/min | 0.7 cm H₂O |
| 60 l/min | 1.8 cm H₂O |
| 90 l/min | 3.60 cm H₂O |
| Bacterial filtration efficiency | 99.99% |
| Viral filtration efficiency | 99.99% |
| Dead space (connectors | 35 ml |
| included -- ISO 9360 | |
| Weight | 19 g |
| Type of filtration | Electrostatic |

Breathing assistance system 14 may also include any other suitable components for providing breathing assistance to patient 12. For example, breathing assistance system 14 may include one or more sensors for sensing, detecting, and/or monitoring one or more parameters related to system 10 and/or patient 12, a control system for controlling gas delivery system 20, various user interfaces, and a display.

Connection system 16 may be generally configured to deliver gas from breathing assistance system 14 to patient 12 and/or to remove exhaust gas away from patient 12. For example, connection system 16 may comprise any suitable type of breathing circuit (e.g., a single-limb or dual-limb circuit) and/or a patient connection apparatus. A patient connection apparatus may include any device or devices configured to connect the breathing circuit to one or more breathing passageways of patient 12. For example, a patient connection apparatus may include a patient connection tube directly connected to the patient's trachea, an artificial airway (e.g., an endotracheal tube or other device) inserted in the patient's trachea, and/or a mask, cushion or nasal pillows positioned over the patient's nose and/or mouth. Connection system 16 may be directly or indirectly coupled to breathing assistance system 14 in any suitable manner. For example, connection system 16 may be coupled directly or indirectly to outlet 36 of humidifier system 22.

FIGS. 2A through 5 illustrate various example configurations of breathing assistance system 14 for use in system 10 discussed herein.

FIG. 2A illustrates an example breathing assistance system 14 including a microporous filter 24 located between gas delivery system 20 and humidifier system 22, in an upright operational orientation, according to certain embodiments of the disclosure. In particular, microporous filter 24 may be located between a gas outlet 40 of gas delivery system 20 and a gas inlet 42 of humidifier system 22. Filter 24 may be housed in a unitary component that couples gas delivery system 20 with gas inlet 42 of humidifier system 22. Alternatively, filter 24 may be coupled between gas outlet 40 of gas delivery system 20 and gas inlet 42 of humidifier system 22 via one or more suitable conduits 44. As shown in FIG. 2A, a gas flow 50 generated by gas flow generation device 30 may flow through gas outlet 40 of gas delivery system 20, through microporous filter 24, through gas inlet 42 of humidifier system 22 and into liquid water chamber 34, become humidified by liquid water in chamber 34, and then flow out through outlet 36 towards patient 12. In some embodiments, the humidification may be promoted by a heater 38. Having microporous filter 24 separate from gas delivery system 20 and humidifier system 22 may allow for relatively easy installation, monitoring, cleaning, and/or replacement of filter 24.

FIG. 2B illustrates the breathing assistance system 14 of FIG. 2A in an upside-down orientation, wherein humidifier system 22 is located above gas delivery system 20, e.g., during transportation of system 14 or where system 14 has fallen off a table or other support. In this upside-down orientation, the water in liquid water chamber 34 may flow against microporous filter 24 (due to gravity), but microporous filter 24 acts as a liquid barrier to substantially prevent the water from entering into gas delivery system 20 and potentially damaging components of gas flow generation device 30. In some configurations, some or all of the liquid water may flow out of humidifier system 22 through outlet 36. In other configurations, liquid water chamber 34 may be shaped (e.g., with twists and turns or labyrinth structures) to substantially prevent liquid water from escaping out through outlet 36 when system 14 is turned in different orientations.

FIG. 3A illustrates an example breathing assistance system 14 including a microporous filter 24 located in gas delivery system 20, in an upright operational orientation, according to certain embodiments of the disclosure. In such embodiments, humidifier system 22 is integrated with gas delivery system 20, and microporous filter 24 is located at the boundary between gas delivery system 20 and humidifier system 22. Thus, as shown in FIG. 3B, filter 24 may prevent liquid water from entering into gas delivery system 20 when system 14 is tilted, turned on its side, or turned upside-down. In other configurations, filter 14 is located further within gas delivery system 20, such that when system 14 is tilted, turned on its side, or turned upside-down, liquid water may enter partially into gas delivery system 20 but is prevented by filter 24 from entering gas flow generation device 30. In still other configurations, filter 14 is located partially but not fully within gas delivery system 20.

As shown in FIG. 3A, a gas flow 50 generated by gas flow generation device 30 may flow through microporous filter 24 and into liquid water chamber 34 of humidifier system 22, become humidified by liquid water in chamber 34, and then flow out through outlet 36 towards patient 12. In some embodiments, the humidification may be promoted by a heater 38.

FIG. 3B illustrates the breathing assistance system 14 of FIG. 3A in an upside-down orientation, wherein humidifier system 22 is located above gas delivery system 20, e.g., during transportation of system 14 or where system 14 has fallen off a table or other support. The water in liquid water chamber 34 may flow against microporous filter 24 (due to gravity), but microporous filter 24 acts as a liquid barrier to substantially prevent the water from entering into gas delivery system 20 and potentially damaging components of gas flow generation device 30.

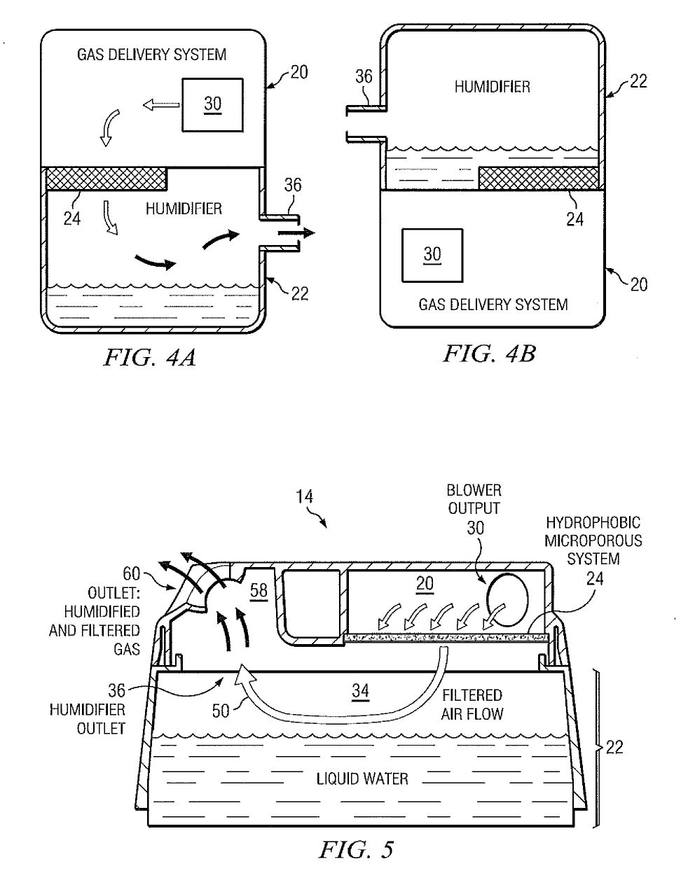

FIG. 4A illustrates an example breathing assistance system 14 including a microporous filter 24 located in humidifier system 22, in an upright operational orientation, according to certain embodiments of the disclosure. In such embodiments, humidifier system 22 is integrated with gas delivery system 20, and microporous filter 24 is located at the boundary between gas delivery system 20 and humidifier system 22. Thus, as shown in FIG. 4B, filter 24 may prevent liquid water from entering into gas delivery system 20 when system 14 is tilted, turned on its side, or turned upside-down. In other configurations, filter 14 is located further within humidifier system 22. In still other configurations, filter 14 is located partially but not fully within humidifier system 22.

As shown in FIG. 4A, a gas flow 50 generated by gas flow generation device 30 may flow through microporous filter 24 and into liquid water chamber 34 of humidifier system 22, become humidified by liquid water in chamber 34, and then flow out through outlet 36 towards patient 12. In some embodiments, the humidification may be promoted by a heater 38.

FIG. 4B illustrates the breathing assistance system 14 of FIG. 4A in an upside-down orientation, wherein humidifier system 22 is located above gas delivery system 20, e.g., during transportation of system 14 or where system 14 has fallen off a table or other support. The water in liquid water chamber 34 may flow against microporous filter 24 (due to gravity), but microporous filter 24 acts as a liquid barrier to substantially prevent the water from entering into gas delivery system 20 and potentially damaging components of gas flow generation device 30.

FIG. 5 illustrates an example breathing assistance system 14 including an integrated gas delivery system 20 and humidifier system 22, according to certain embodiments of the disclosure. The top portion of system 14 includes gas delivery system 20 and a delivery chamber 58 having an outlet for connection to a patient circuit 16. The bottom of system 14 comprises a humidifier system 22 including a liquid water chamber 34. The top portion of system 14 (including gas delivery system 20 and delivery chamber 58) may be removable from humidifier system 22, or may be permanently attached.

A microporous filter 24 separates gas delivery system 20 from humidifier system 22. Gas delivery system 20 includes a blower 30 that delivers a pressurized air flow 50 through microporous filter 24 and into liquid water chamber 34, where the air flow is humidified. The humidified air then flows through outlet 36 of humidifier system 22, into delivery chamber 58, and out though outlet 60 toward patient 12 (e.g., via a patient circuit connected to outlet 60). Microporous filter 24 prevents liquid water in chamber 34 from flowing into gas delivery system 20 (and potentially damaging blower 30 and other components) if system 14 is tilted, turned on its side, or turned upside-down.

FIG. 6 illustrates a method for protecting a gas delivery system 30 from water damage using a microporous filter 24, according to certain embodiments of the disclosure.

At step 100, a breathing assistance system 14 oriented in an upright position is operated to deliver humidified gas toward a patient 12. In particular, gas flow generation device 30 generates a gas flow, which is communicated through the microporous filter 24, and into a liquid water chamber 34 of a humidifier system 22, where the gas flow is humidified. The humidified gas flows out through an outlet 36 of humidifier system 22, and toward patient 12.

At step 102, either during or after operation of system 14, system 14 is moved (e.g., tilted, turned, or flipped over) to an orientation in which liquid water from liquid water chamber 34 flows against microporous filter 24, which physically prevents the liquid water flowing into the gas flow generation device 30, thereby protecting device 30 from damage by the water. In some configurations, some or all of the water may flow out through the humidifier system outlet 36.

It will be appreciated that while the disclosure is particularly described in the context of breathing assistance systems, the apparatuses, techniques, and methods disclosed herein may be similarly applied in other contexts. Additionally, it should be understood that various changes, substitutions and alterations can be made herein without departing from the spirit and scope of the disclosure as illustrated by the following claims.

What is claimed is:

1. A breathing assistance system, comprising:
a gas delivery system including a gas flow generation device configured to generate a gas flow;
a humidifier system including a liquid water chamber and an outlet, the gas delivery system and the humidifier system at least partially defining a gas flow path from the gas flow generation device to the outlet of the humidifier system; and
a microporous filter located along the gas flow path such that the gas flow generated by the gas flow generation device flows through the microporous filter, subsequently becomes humidified in the liquid water chamber, and subsequently flows through the outlet of the humidifier system;
wherein the microporous filter is gas-permeable and liquid water-impermeable, such that the microporous filter prevents liquid water in the liquid water chamber from flowing into the gas flow generation device in any orientation of the gas delivery system and humidifier system.

2. A breathing assistance system according to claim 1, wherein the microporous filter comprises a copolymer filter.

3. A breathing assistance system according to claim 2, wherein the microporous filter comprises a polypropylene filter.

4. A breathing assistance system according to claim 1, wherein the microporous filter comprises an anti-bacterial membrane.

5. A breathing assistance system according to claim 1, wherein:
the microporous filter is located between a gas outlet of the gas delivery system and a gas inlet of the humidifier system; and
the filter prevents liquid water in the liquid water from flowing into the gas delivery system.

6. A breathing assistance system according to claim 1, wherein the microporous filter is located at least partially within the gas delivery system.

7. A breathing assistance system according to claim 1, wherein the microporous filter is located at least partially within the humidifier system.

8. A breathing assistance system according to claim 1, wherein the humidifier system is physically integrated with the gas delivery system.

9. A breathing assistance system according to claim 1, wherein the breathing assistance system comprises a portable CPAP device or a portable ventilator.

10. A breathing assistance system according to claim 1, further comprising a patient circuit connected between the outlet of the humidifier system and a patient, the patient circuit configured to deliver humidified gas toward the patient.

11. A method for providing humidification for a breathing assistance system including a gas delivery system including a gas flow generation device, a humidifier system including a liquid water chamber and an outlet, and a gas-permeable, liquid water-impermeable microporous filter located along a gas flow path from the gas flow generation device to the humidifier system outlet, the microporous filter being permeable to gas but impermeable to liquid water, the method comprising:
generating a gas flow using the gas flow generation device;
communicating the gas flow from the gas flow generation device, through the gas-permeable, liquid water-impermeable microporous filter, and into the liquid water chamber;
humidifying the gas flow in the liquid water chamber; and
communicating the humidified gas flow through the humidifier system outlet.

12. A method according to claim 11, further comprising:
operating the breathing assistance system at a first physical orientation of the breathing assistance system; and
moving the breathing assistance system to a second physical orientation in which the microporous filter physically prevents liquid water in the liquid water from flowing into the gas flow generation device.

13. A method according to claim 11, wherein moving the breathing assistance system to a second physical orientation comprises turning the breathing assistance system substantially on its side or upside-down.

14. A method according to claim 11, further comprising delivering the humidified gas flow toward a patient via a patient circuit connected between the humidifier system outlet and the patient.

15. A method according to claim 14, comprising using the humidified gas flow for CPAP or ventilation treatment to the patient.

16. A breathing assistance system, comprising:
a gas flow generation device configured to generate a gas flow;
a humidifier; and
a microporous filter located between the gas flow generation device and the humidifier such that the gas flow generated by the gas flow generation device flows through the microporous filter, subsequently becomes humidified in a chamber of the humidifier, and subsequently flows through an outlet of the humidifier;
wherein the microporous filter is gas-permeable and liquid water-impermeable, such that the microporous filter prevents liquid in the humidifier from flowing to the gas flow generation device in any orientation of the breathing assistance system.

17. A breathing assistance system according to claim 15, wherein the microporous filter comprises a polypropylene filter.

18. A breathing assistance system according to claim 15, wherein the microporous filter is located at least partially within the humidifier.

19. A breathing assistance system according to claim 15, wherein the humidifier and gas flow generation device are housed in a physically integrated housing.

20. A breathing assistance system according to claim 15, further comprising a patient circuit connected between the outlet of the humidifier and a patient, the patient circuit configured to deliver humidified gas toward the patient.
